(19) United States
(12) Patent Application Publication
(10) Pub. No.: US 2024/0120042 A1
(43) Pub. Date: Apr. 11, 2024

(54) LEARNING DEVICE, DETERMINATION DEVICE, METHOD FOR GENERATING TRAINED MODEL, AND RECORDING MEDIUM

(71) Applicant: NEC Corporation, Minato-ku, Tokyo (JP)

(72) Inventor: Yuji Ohno, Tokyo (JP)

(73) Assignee: NEC Corporation, Minato-ku, Tokyo (JP)

(21) Appl. No.: 18/273,481

(22) PCT Filed: Mar. 29, 2021
(86) PCT No.: PCT/JP2021/013209

§ 371 Date: Jul. 20, 2023

(51) Int. Cl. G16H 10/60; G06V 40/20; G16H 20/10; G16H 20/60; G16H 50/30

(52) U.S. Cl. CPC G16H 10/60; G06V 40/20; G16H 20/10; G16H 20/60; G16H 50/30

(57) ABSTRACT

This learning device comprises: an acquisition unit for acquiring biometric information of a patient and medical chart information of the patient; a selection unit for selecting, on the basis of the medical chart information, the biometric information of the patient that allows the determination of whether the patient is in agitation; and a model generation unit for generating an agitation determination model for determining whether a subject patient is in agitation, by using the selected biometric information and on the basis of the biometric information of the subject patient.

### TECHNICAL FIELD

The present invention relates to a learning device, a determination device, a method for generating a trained model, and a recording medium.

### BACKGROUND ART

There is a possibility that a patient falls into an agitated state in a medical or care field. When the patient falls into an agitated state, the risk of decannulation, needle withdrawal, removal, tumbling, falling, or the like increases, and there is a possibility that the patient is injured. Therefore, a technique for detecting such a patient's agitated state in advance is known.

PTL 1 discloses a biometric information processing system that determines, based on a feature amount of input biometric information of a target patient, identification information indicating whether a condition of the target patient has changed in comparison with a normal state, and estimates countermeasure information for the target patient based on the identification information and countermeasure prediction parameters that are preliminarily learned.

### CITATION LIST

### Patent Literature

PTL 1: WO 2019/073927 A1

### SUMMARY OF INVENTION

### Technical Problem

It is necessary to accurately determine whether the patient is in an agitated state in order to reduce the risk of the patient's decannulation, needle withdrawal, removal, tumbling, falling, or the like. In order to accurately determine whether of being in an agitated state, it is preferable to increase the accuracy of a model for determining the agitated state disclosed in PTL 1.

Therefore, the present invention has been made to solve the above problem, and an object is to provide a device and the like capable of improving the accuracy of a model for determining a patient's state.

### Solution to Problem

A learning device according to an aspect of the present invention includes an acquisition means that acquires biometric information of a patient and medical record information of the patient, a selection means that selects, based on the medical record information, the biometric information of the patient from which it is possible to determine whether of being in an agitated state, and a model generation means that generates an agitation determination model for determining whether a target patient is in an agitated state based on the biometric information of the target patient by using the selected biometric information.

A determination device according to an aspect of the present invention includes a determination means that determines whether the target patient is in the agitated state by using the biometric information of the target patient and the agitation determination model, in which the agitation determination model is a trained model generated by a learning device including an acquisition means that acquires biometric information of a patient and medical record information of the patient, a selection means that selects, based on the medical record information, the biometric information of the patient from which it is possible to determine whether of being in the agitated state, and a model generation means that generates an agitation determination model for determining whether a target patient is in an agitated state based on the biometric information of the target patient by using the selected biometric information.

A method for generating a trained model according to an aspect of the present invention includes, by a computer acquiring biometric information of a patient and medical record information of the patient, selecting the biometric information of the patient from which it is possible to determine whether of being in an agitated state by using the medical record information, and generating an agitation determination model for determining whether a target patient is in the agitated state based on the biometric information of the target patient by using the selected biometric information.

A recording medium according to an aspect of the present invention stores a program for causing a computer to execute processing of acquiring biometric information of a patient and medical record information of the patient, selecting the biometric information of the patient from which it is possible to determine whether of being in an agitated state by using the medical record information, and generating an agitation determination model for determining whether a target patient is in the agitated state based on the biometric information of the target patient by using the selected biometric information.

### Advantageous Effects of Invention

According to the present invention, it is possible to improve the accuracy of the model for determining the patient's state.

### BRIEF DESCRIPTION OF DRAWINGS

FIG. 1 is a block diagram illustrating a configuration of a learning device 10 according to a first example embodiment.

FIG. 2 is a flowchart illustrating a flow of operation performed by the learning device 10 according to the first example embodiment.

FIG. 3 is a block diagram illustrating a configuration of an agitation determination system 200 according to a second example embodiment.

FIG. 4 is a flowchart illustrating a flow of operation performed by the agitation determination system 200 according to the second example embodiment.

FIG. 5 is a block diagram illustrating an example of a hardware configuration.

### EXAMPLE EMBODIMENT

Hereinafter, each example embodiment of the present invention will be described with reference to the drawings.

In each example embodiment of the present invention, an agitation determination model is a trained model that determines whether the patient is in an agitated state. The agitated state indicates a state in which the patient is in agitation. The agitated state may include a state in which the mental cannot be normally controlled. The agitated state may include a state caused by delirium of the patient. The agitated state may be caused by a mental or physical factor of the patient. It has been found that the patient often causes a problematic behavior when in an agitated state. That is, the patient in an agitated state is highly likely to cause a problematic behavior. Accordingly, by grasping whether the patient is in an agitated state, it is possible to predict whether the patient is likely to cause a problematic behavior. Here, the problematic behavior of the patient is, for example, behavior that requires some measure by a medical worker who performs a medical treatment action on the patient in response to the behavior. The problematic behavior of the patient is, for example, leaving the bed, walking alone, loitering, going to another floor of the hospital, removing the fence of the bed, falling out of the bed, fiddling with a drip or tubing, removal of a drip or tubing, making a strange voice, making abusive language, use of violence, or the like. Whether the behavior of the patient falls under the problematic behavior may be determined according to the condition of the patient to be described below. In each example embodiment of the present invention, the agitation determination model may determine whether the patient is causing a problematic behavior. Hereinafter, a normal state of the patient, that is, a state that is not the agitated state is referred to as a non-agitated state.

In each example embodiment of the present invention, a patient is a person who receives a medical treatment action from a medical worker. The patient is not limited to this as long as it is a subject to be determined for the agitated state.

In each example embodiment of the present invention, the biometric information is information that changes with the life activity of the patient. That is, the biometric information is time-series information indicating a change associated with the life activity of the patient. The biometric information is, for example, at least one of a heart rate, heart rate variability, a respiratory rate, a blood pressure value, a body temperature, a skin temperature, a blood flow rate, a blood oxygen saturation, a body motion, and the like. The biometric information may include other information used for determining an agitated state. The biometric information is measured using, for example, at least one sensor attached to the patient. The sensor is, for example, a heart rate sensor, a respiration rate sensor, a blood pressure sensor, a body temperature sensor, a blood oxygen saturation sensor, an acceleration sensor, or the like. The patient may wear a device on which one sensor is mounted or wear a device on which a plurality of sensors is mounted. The patient may wear a plurality of devices. The device is mainly a wearable device, and specific examples include a smart watch, a smart band, an active tracker, a clothing sensor, a wearable heart rate sensor, and the like. The biometric information may be extracted from, for example, one or both of image information acquired by an imaging device (camera or the like) installed in a patient's hospital room and sound information of a patient's voice and a surrounding environment of the patient.

### First Example Embodiment

Hereinafter, a configuration of the learning device 10 according to the first example embodiment will be described. The learning device 10 according to the present example embodiment generates an agitation determination model.

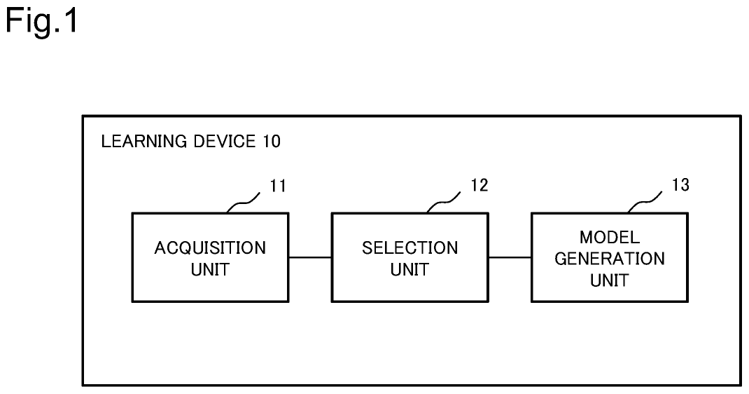

FIG. 1 is a block diagram illustrating a configuration of the learning device 10 according to the present example embodiment. The learning device 10 illustrated in FIG. 1 includes an acquisition unit 11, a selection unit 12, and a model generation unit 13.

The acquisition unit 11 is an acquisition means that acquires biometric information of a patient and medical record information of the patient.

As an example, the biometric information is stored in a storage device, which is not illustrated, or the like in association with a patient ID for identifying a patient in association with time information indicating a time when the biometric information is measured. The acquisition unit 11 may acquire the biometric information of the patient from the storage device. The acquisition unit 11 may acquire the biometric information of the patient associated with the time information from the sensor or the device communicably connected to the learning device 10 via a communication network such as wireless or wired.

The medical record information is information described in the medical record of the patient. The medical record information includes at least one of patient basic information, information regarding a condition, and information regarding a medical treatment action. The patient basic information is, for example, information associated with a health insurance card of the patient or information regarding the patient obtained from a medical interview sheet. The patient basic information is, as a more detailed example, a patient name, an age, a sex, a medical history, a family history, a social history, or the like, but may include other information.

The information regarding the condition is information indicating the medium- to long-term state of the patient. The information regarding the condition includes, for example, information regarding at least one state of cognitive function, physical function, and motor function of the patient. The information regarding the condition includes, for example, at least one of a disease name, a disease state, a Glasgow Coma Scale (GCS) score, a Japan Coma Scale (JCS) score, a Manual Muscle Test (MMT) score, and the like of the patient. The information regarding the condition is not limited to this as long as it is information that serves as an index for the medical worker to determine the condition of the patient. The information regarding the condition may include, for example, at least one score of the level of care needed, functional independence measure (FIM), stroke impairment assessment set (SIAS), or Berg balance scale (BBS).

The information regarding the medical treatment action is information recording a medical treatment action performed on the patient. The medical treatment action includes, for example, a medical examination action performed by a doctor, medical treatment care and medical examination assistance that are actions performed by a nurse. Hereinafter, the information regarding the medical treatment action is described as a medical treatment record as an example, but is not limited thereto. The medical treatment record includes at least one of a medication record, a treatment record, a restraint record, a meal record, and the like. The medication record is a record regarding medication of the patient. The medication record includes, for example, the type and amount of medicine taken by the patient, the time when the patient took the medicine, and the like. The treatment record is a record regarding the treatment performed on one or both of the patient and the surrounding environment of the patient. The treatment record includes, for example, the type of treatment, the time when the treatment was performed, and the like. Specifically, the treatment is change of clothes, change of diapers, position change for preventing bedsores, bed making, weight measurement, insertion and removal of drips, and the like. The restraint record is a record regarding restraint performed on the patient. Here, the restraint includes physical restraint of the patient. The restraint record includes a type of restraint, a restrained site, a time when the restraint was performed, and the like. The meal record is a record regarding a meal taken by the patient. The meal record includes a meal content, a meal time, a meal amount, and the like.

The medical record information is acquired, for example, by a medical worker performing recording on the medical record of the patient. The medical record information may include information recorded by the medical worker on those other than the medical record regarding the patient. The medical record information may be extracted and acquired from, for example, one or both of image information acquired by the camera installed in a patient's hospital room and sound information of a patient's voice and a surrounding environment of the patient.

The medical record information is stored in a storage device, which is not illustrated, or the like in association with the patient ID. The acquisition unit 11 may acquire the medical record information of the patient from the storage device, or may directly acquire the medical record information of the patient from an input device to which the medical record information is input by the medical worker, which is communicably connected to the learning device 10 using wireless communication, by wire, or the like.

The acquisition unit 11 may acquire the medical record information each time the medical record information is updated. For example, the condition of the patient may not change over a period of weeks to months, and in this case, there is a possibility that update is not performed. In this case, the acquisition unit 11 acquires information regarding the condition of the patient at a pace of once every several weeks to several months. Since it is conceivable that the information regarding the medical treatment action is updated each time the medical worker performs recording on the medical record, the acquisition unit 11 acquires the information regarding the medical treatment action each time the medical record is updated. That is, the acquisition unit 11 may acquire only the updated information within the medical record information.

The selection unit 12 is a selection means that selects biometric information from which it is possible to determine whether the patient is in an agitated state based on the medical record information. Specifically, the selection unit 12 selects biometric information of the patient in which the information included in the medical record information acquired by the acquisition unit 11 satisfies one or both of a first condition and a second condition, as biometric information of the patient from which it is possible to determine whether of being in an agitated state.

The biometric information of the patient selected by the selection unit 12 is labeled as to an agitated state or a non-agitated state, which will be described below. In the selection unit 12, the biometric information of the patient from which it is possible to determine whether of being in an agitated state by the labeling is selected. That is, the biometric information of the patient selected by the selection unit 12 may be biometric information that is relatively easily labeled as to whether of being in an agitated state.

The selection unit 12 selects biometric information of the patient in which the information regarding the condition of the patient satisfies the first condition regarding the condition of the patient, as the biometric information of the patient from which it is possible to determine whether of being in an agitated state. As described above, the information regarding the condition is information included in the medical record information. The first condition is a condition regarding the degree of the condition of the patient.

For example, the selection unit 12 selects, for example, biometric information related to a patient having a low severity of the condition based on the first condition. The biometric information selected based on the first condition is, for example, biometric information related to a patient having a GCS score of equal to or more than 9, a JCS score of equal to or less than 11, an MMT score of equal to or more than 2, or the like. The GCS score is an index for determining the level of consciousness of a brain surgery patient, and is generally classified into three stages: mild, moderate, and severe. The GCS score is determined based on an observation result in three items: E (eye opening), V (verbal response), and M (motor response). A patient having a GCS score of equal to or more than 9 is a patient classified as mild or moderate. The JCS score is classified according to the degree of awakening, and is also called 3-3-9 degree method in terms of the way of classification, and the larger the numerical value, the more severe the consciousness disorder is. In the JCS score, a state of awakening without stimulation is expressed by a single-digit number, a state of awakening with stimulation is expressed by a double-digit number, and a state of not awakening with stimulation is expressed by a three-digit number. Patients having a JCS score of equal to or less than 11 include patients who show a state of being easily awakened by a call, or a state of being almost lucid but not clear even without stimulation. The MMT score is an index for determining the degree of paralysis of the limbs of the brain surgery patient, and is evaluated in six grades of scores 0 to 5. The patient having an MMT score of equal to or more than 2 includes a normal patient to a patient who can completely move when gravity is removed.

The selection unit 12 may select the biometric information related to the patient capable of conscious movement or utterance based on of the first condition.

As described above, when performing selection based on the first condition, the selection unit 12 uses the information regarding the condition of the patient included in the medical record information. The selection unit 12 selects the biometric information of the patient using the information related to the determined first condition within the information regarding the condition of the patient included in the medical record information acquired by the acquisition unit 11.

The selection unit 12 selects biometric information of the patient in which the information regarding the medical treatment action satisfies the second condition regarding the medical treatment action as the biometric information of the patient from which it is possible to determine whether of being in an agitated state. As described above, the information regarding the medical treatment action is information included in the medical record information. The second condition is a condition regarding the degree of variation in the biometric information of the patient caused by the medical treatment action. The biometric information selected based on the second condition is, for example, biometric information in a time other than the time when the medical treatment action is performed on the patient.

When performing selection based on the second condition, the selection unit 12 uses the information regarding the medical treatment action on the patient included in the medical record information. The selection unit 12 selects the biometric information of the patient using the information related to the determined second condition within the information regarding the medical treatment action on the patient included in the medical record information acquired by the acquisition unit 11.

Here, an example of the biometric information selected based on the second condition will be described. As an example, the biometric information selected based on the second condition includes biometric information after a predetermined time has elapsed since the medicine was taken. The predetermined time is, for example, an effect holding time of the taken medicine, or the like. This is because there is a possibility that the biometric information of the patient who has taken the medicine unnaturally varies or is stabilized due to the action of the medicine during the time when the effect of the medicine is held. The effect holding time of the medicine is specified, for example, by collating the type and amount of the medicine taken by the patient included in the medical treatment record with a database or the like including at least the type and amount of the medicine and the effect holding time. In this case, the selection unit 12 refers to the information regarding medication included in the information regarding the medical treatment action. The selection unit 12 refers to the time information indicating the time when the biometric information was measured, and selects the biometric information at the time when the effect holding time specified from the type and amount of the medicine taken by the patient has elapsed from the time when the medicine included in the information regarding the medication was taken.

As an example, the biometric information selected based on the second condition includes biometric information at a time other than the time when the treatment was performed, biometric information at a time other than the time when the restraint was performed, and the like. This is because, for the time during which the treatment or restraint is performed on the patient or the time during which the treatment is performed on the surrounding environment of the patient, there is a possibility that the biometric information of the patient unnaturally varies due to occurrence of movement irrelevant to the patient's intention or restriction of the patient's voluntary movement due to the treatment or restraint. In this case, for example, the selection unit 12 refers to the information regarding the treatment included in the information regarding the medical treatment action according to the second condition. The selection unit 12 may refer to the information regarding the restraint instead of the information regarding the treatment or together with the information regarding the treatment. The selection unit 12 refers to the time information indicating the time when the biometric information was measured, and selects the biometric information at the time not falling under the time when the treatment was performed. The selection unit 12 refers to the time information indicating the time when the biometric information was measured, and selects the biometric information at the time not falling under the time when the restraint was performed.

Further, as an example, the biometric information selected based on the second condition includes biometric information after a predetermined time from a meal. This is because there is a possibility that the biometric information of the patient unnaturally varies due to movement of the patient accompanying the meal or a biometric reaction accompanying digestion during the meal and within a predetermined time after the end of the meal. In this case, the selection unit 12 refers to the information regarding meal included in the information regarding the medical treatment action. The selection unit 12 refers to the time information indicating the time when the biometric information was measured, and selects the biometric information of the time when a predetermined time has elapsed from the meal time included in the medical treatment record. The predetermined time described above is, for example, 30 minutes, but is not limited thereto, and it is sufficient if it is appropriately determined.

The selection unit 12 may select the biometric information of the patient to be used for training of the agitation determination model based on either one of the first condition and the second condition, or may select the biometric information based on both the first condition and the second condition. In a case where the biometric information is selected based on both the first condition and the second condition, the selection unit 12 may select biometric information from which it is possible to determine whether of being in an agitated state by applying the second condition to the biometric information selected by applying the first condition.

The model generation unit 13 is a model generation means that generates an agitation determination model using the selected biometric information. Here, the agitation determination model is a model that determines whether the patient is in an agitated state based on the biometric information of the patient. Hereinafter, the patient subjected to agitation determination may be referred to as a target patient. The model generation unit 13 performs machine learning using the biometric information labeled as to an agitated state or a non-agitated state as training data, and generates an agitation determination model.

As described above, the agitation determination model is a model that determines whether the patient is in an agitated state based on the biometric information of the patient. The agitation determination model outputs an agitation score using the biometric information of the patient as an input. The agitation score is a value serving as an index indicating an agitated state or a non-agitated state. The agitation score is, for example, a value of equal to or more than 0 and equal to or less than 1. In this case, an agitation score closer to 1 indicates a higher possibility of being in an agitated state, and an agitation score closer to 0 indicates a higher possibility of being in a non-agitated state. For example, a preset value of equal to or more than 0 and equal to or less than 1 is used as a threshold, and an agitated state or a non-agitated state is determined with reference to the threshold. The agitation score may be a value expressed by a binary value of 0 or 1. In this case, the agitation score indicates 1 in an agitated state, and indicates 0 in a non-agitated state.

The model generation unit 13 performs training using, for example, a support vector machine (SVM), a neural network, other known machine learning methods, or the like, using the biometric information labeled as to an agitated state or a non-agitated state as training data.

Labeling as to an agitated state or a non-agitated state with respect to the biometric information selected by the selection unit 12 may be performed by the model generation unit 13, or may be performed by another device, which is not illustrated, or a user.

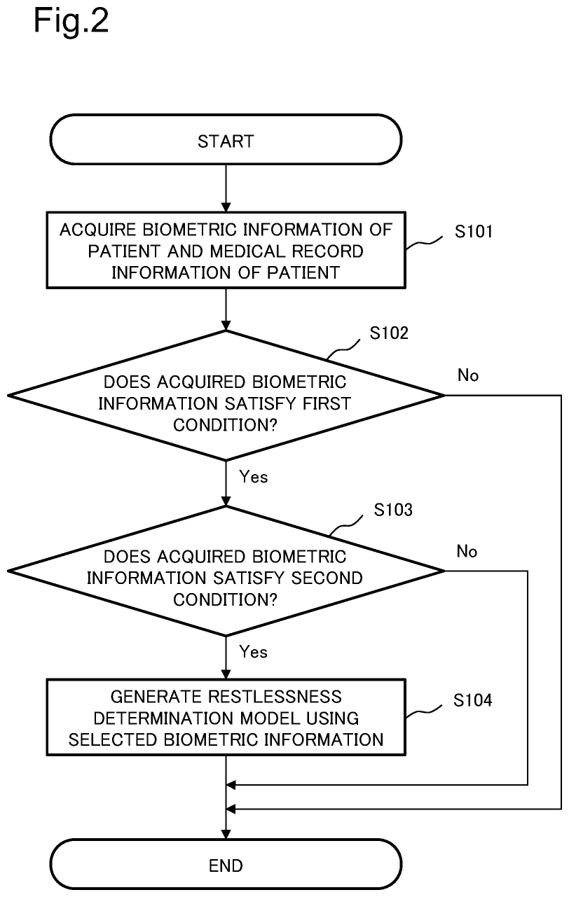

Next, operation performed by the learning device 10 will be described with reference to FIG. 2. FIG. 2 is a flowchart illustrating an example of operation performed by the learning device 10.

The acquisition unit 11 acquires the biometric information of the patient and the medical record information of the patient (step S101).

The selection unit 12 selects the biometric information of the patient from which it is possible to determine whether of being in an agitated state using the medical record information from steps S102 to S103. First, the selection unit 12 selects the biometric information of the patient satisfying the first condition regarding the condition of the patient by using the information regarding the condition of the patient included in the medical record information (step S102). In a case where the acquired biometric information satisfies the first condition (step S102: Yes), the selection unit 12 further selects the biometric information of the patient satisfying the second condition regarding the medical treatment record by using the medical treatment record of the patient included in the medical record information (step S103). In step S103, in a case where the acquired biometric information does not satisfy the first condition (step S102: No) or in a case where the acquired biometric information does not satisfy the second condition (step S103: No), the learning device 10 does not use the biometric information as the training data and ends the processing. When the acquired biometric information satisfies the second condition (step S103: Yes), the model generation unit generates an agitation determination model using the biometric information selected by the selection unit 12 (step S104).

The operation of the learning device 10 described above indicates an example in a case where both the selection of the biometric information based on the first condition and the selection of the biometric information based on the second condition are performed in the selection unit 12. In this case, the selection of the biometric information based on the first condition (step S102) and the selection of the biometric information based on the second condition (step S103) may be reordered. In a case where either the selection of the biometric information based on the first condition or the selection of the biometric information based on the second condition is not performed, the operation of either step S103 or step S102 is omitted. The operation of the learning device 10 described above may be performed, for example, when a predetermined number or more of pieces of biometric information is accumulated in a storage device, which is not illustrated, or the like.

In the agitation determination model, training is performed using training data in which labeling as to whether of being in an agitated state is accurately performed with respect to the collected biometric information of the patient, which leads to improvement in accuracy. However, there is a possibility that the collected biometric information of the patient includes, for example, biometric information of patients with various severities, biometric information when the patient is receiving a medical treatment action, and the like. When the biometric information is collected regardless of such a patient's state or situation, it may be difficult to accurately label the collected biometric information as to whether of being in an agitated state. For this reason, there is a possibility that the agitation determination model is trained using the training data in which the biometric information from which it is unclear whether of being in an agitated state is labeled, and it may be difficult to generate a highly accurate agitation determination model. The learning device 10 according to the present example embodiment selects the biometric information of the patient from which it is possible to determine whether of being in an agitated state based on the medical record information in the selection unit 12. Thus, when labeling as to an agitated state or a non-agitated state is performed with respect to the biometric information, the learning device 10 can extract the training data with which there is a possibility that it is easy to determine an agitated state or a non-agitated state. Then, the learning device 10 generates the agitation determination model using the biometric information selected by the selection unit 12 in the model generation unit 13. With such a configuration, the learning device 10 can perform training using only the accurately labeled biometric information as training data, and can generate an accurate agitation determination model.

The learning device 10 according to the present example embodiment selects the biometric information related to a patient having a low severity of the condition based on the first condition in the selection unit 12 as described above. When the severity of the condition of the patient is high, anomaly appears in the biometric information of the patient, and when labeling as to an agitated state or a non-agitated state is performed with respect to the biometric information, there is a possibility that it is difficult to determine an agitated state or a non-agitated state. For this reason, when the selection unit 12 selects the biometric information related to the patient having a low severity of the condition based on the first condition, the learning device 10 can extract the biometric information of the patient from which it is possible to determine an agitated state or a non-agitated state. Thus, the learning device 10 can use training data in which labeling as to an agitated state or a non-agitated state is accurately performed with respect to the biometric information. Further, the learning device 10 can generate an accurate agitation determination model by performing training using the accurately labeled biometric information as training data.

The learning device 10 according to the present example embodiment selects the biometric information related to a patient capable of conscious movement or utterance based on of the first condition in the selection unit 12 as described above. When the patient is not capable of conscious movement or utterance, even when the patient is in an agitated state, the patient does not express it in an action or speech and behavior, and there is a high possibility that it is difficult to determine whether the patient is in an agitated state. For this reason, when the selection unit 12 selects the biometric information related to the patient capable of conscious movement or utterance based on the first condition, the learning device 10 can extract the biometric information of the patient from which it is possible to determine an agitated state or a non-agitated state. Thus, the learning device 10 can use training data in which labeling as to an agitated state or a non-agitated state is accurately performed with respect to the biometric information. Further, the learning device 10 can generate an accurate agitation determination model by performing training using the accurately labeled biometric information as training data.

The learning device 10 according to the present example embodiment selects the biometric information in the time other than the time when the medical treatment action is performed on the patient based on the second condition in the selection unit 12 as described above. While a medical treatment action is being performed on the patient or the effect of a medical treatment action performed on the patient continues, the biometric information of the patient unnaturally varies due to the effect of the medical treatment action, and thus, there is a high possibility that it becomes difficult to determine an agitated state or a non-agitated state. For this reason, when the selection unit 12 selects the biometric information in the time other than the time when the medical treatment action is performed on the patient based on the second condition, the learning device 10 can extract the biometric information of the patient from which it is possible to determine an agitated state or a non-agitated state. Thus, the learning device 10 can use training data in which labeling as to an agitated state or a non-agitated state is accurately performed with respect to the biometric information. Further, the learning device 10 can generate an accurate agitation determination model by performing training using the accurately labeled biometric information as training data.

### Second Example Embodiment

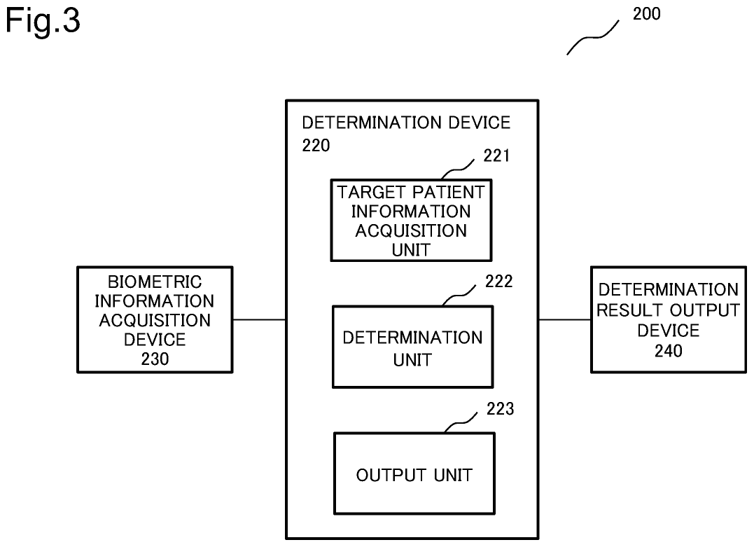

Hereinafter, a configuration of an agitation determination system 200 according to the second example embodiment will be described. FIG. 3 is a block diagram illustrating a configuration of the agitation determination system 200 according to the second example embodiment of the present invention. As illustrated in FIG. 3, the agitation determination system 200 according to the second example embodiment includes a determination device 220, a biometric information acquisition device 230, and a determination result output device 240. The determination device 220 and the biometric information acquisition device 230, and the determination device 220 and the determination result output device 240 are communicably connected to each other by using wireless communication such as Wi-fi or Bluetooth (registered trademark) or by wire.

The determination device 220 determines an agitated state of the target patient using the agitation determination model. The determination device 220 includes a target patient information acquisition unit 221, a determination unit 222, and an output unit 223. The determination device 220 is achieved, for example, in an information terminal such as a computer provided in a medical institution. The determination device 220 may be achieved, for example, on a cloud server.

The target patient information acquisition unit 221 acquires the biometric information of a target patient subjected to determination of an agitated state. The target patient information acquisition unit 221 acquires the biometric information of the target patient by receiving the biometric information acquired by the biometric information acquisition device 230 to be described below and used for determination of an agitated state of the target patient.

The determination unit 222 is a determination means that determines whether the target patient is in an agitated state using the biometric information of the target patient and the agitation determination model. Specifically, the determination unit 222 inputs the biometric information of the target patient to the agitation determination model and obtains an agitation score. Then, the determination unit 222 determines an agitated state or a non-agitated state of the target patient based on the agitation score.

Here, the agitation determination model is a model generated by the learning device 10 in the first example embodiment. That is, the agitation determination model in the present example embodiment is a trained model generated in advance using the biometric information of the patient selected based on the medical record information. The determination unit 222 acquires the agitation determination model stored in a storage device, which is not illustrated, or the like and determines an agitated state of the target patient.

The output unit 223 outputs a determination result of an agitated state of the target patient by the determination unit 222. The output unit 223 outputs the determination result to the determination result output device 240 described below. The output unit 223 outputs the determination result in a format capable of being output by the determination result output device 240. For example, in a case where the determination result output device 240 includes a display means such as a display that outputs a determination result, the output unit 223 has a function as a display control unit that controls the display means. In this manner, the output unit 223 functions as a means that controls the determination result output device 240 according to the format for output of the determination result in the determination result output device 240.

The biometric information acquisition device 230 is a device that acquires the biometric information of the patient. The biometric information acquisition device 230 is, for example, a wearable device or the like. The biometric information acquisition device 230 is, for example, a device including at least one sensor that acquires the biometric information of the patient by being worn on the patient. The biometric information and the sensor are as described above. The biometric information acquisition device 230 may be, for example, an imaging device installed in the patient's hospital room or a device that acquires sound information of the patient's voice and the surrounding environment of the patient. In this case, the biometric information acquisition device 230 performs processing of extracting the biometric information of the patient based on the acquired image information and sound information.

The determination result output device 240 outputs the determination result of an agitated state of the target patient acquired from the determination device 220. The determination result output device 240 is, for example, an information terminal such as a computer provided in a medical institution. The determination result output device 240 may be an information terminal such as a tablet terminal or a smartphone held by a medical worker. The determination result output device 240 includes, for example, at least one of a display means capable of displaying characters and images such as a display, a sound output means capable of outputting a sound such as a speaker, and the like. The determination result output device 240 presents the determination result of an agitated state of the target patient to the medical worker using at least one of the display means, the sound output means, and the like.

The determination result output device 240 may output the biometric information of the target patient acquired by the biometric information acquisition device 230 together with the determination result of an agitated state of the target patient. In this case, the biometric information acquisition device 230 and the determination result output device 240 are communicably connected to each other by using, for example, wireless communication or by wire as described above.

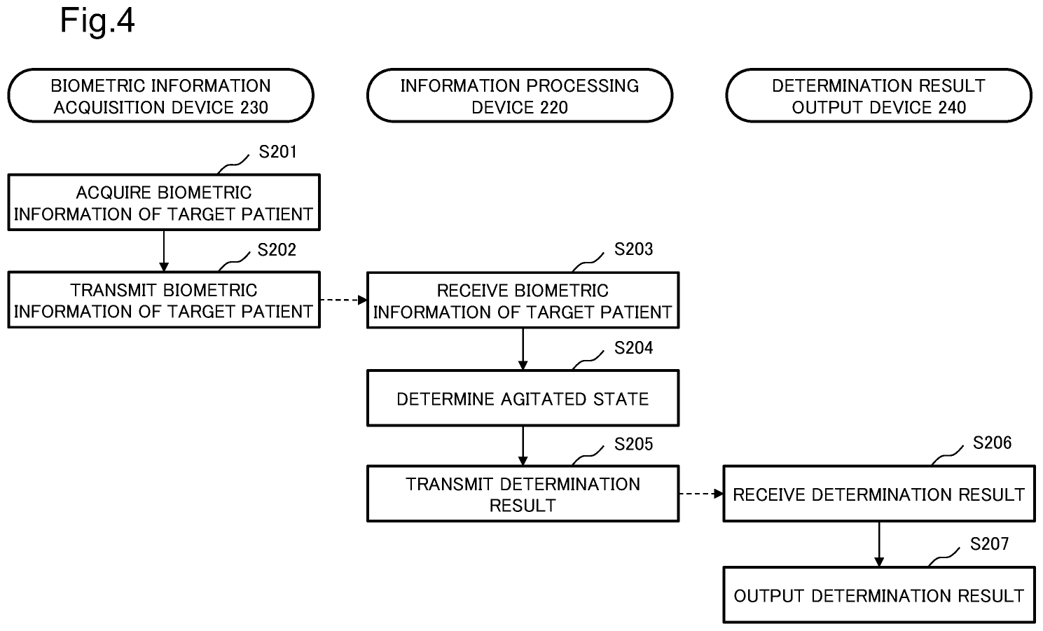

Next, operation performed by the agitation determination system 200 will be described with reference to FIG. 4. FIG. 4 is a flowchart illustrating an example of operation performed by the agitation determination system 200.

The biometric information acquisition device 230 acquires the biometric information of the target patient (step S201). Then, the biometric information acquisition device 230 transmits the acquired biometric information of the subject to the determination device 220 (step S202).

The target patient information acquisition unit 221 of the determination device 220 receives the biometric information of the target patient from the biometric information acquisition device 230 (step S203). The determination unit 222 determines an agitated state of the target patient using the biometric information of the target patient and the agitation determination model (step S204). The output unit 223 transmits the determination result of an agitated state of the target patient by the determination unit 222 to the determination result output device 240 (step S205).

The determination result output device 240 receives the determination result of an agitated state of the target patient from the determination device 220 (step S206). Then, the determination result output device 240 outputs the determination result of an agitated state of the target patient to the medical worker or the like using at least one of the display means, the sound output means, and the like (step S207).

The agitation determination system 200 according to the present example embodiment determines an agitated state of the target patient using the biometric information of the target patient and the agitation determination model in the determination device 220. The agitation determination model is a model generated by the learning device 10 in the first example embodiment. The determination device 220 can accurately determine an agitated state of the target patient by using the agitation determination model. When the determination result in which an agitated state of the target patient is accurately determined is output to the determination result output device 240, the medical worker or the like can efficiently grasp the agitated state of the patient. In this manner, the agitation determination system 200 contributes to improvement of work efficiency of the medical worker and the like.

The agitation determination system 200 may include the learning device 10 according to the first example embodiment. That is, the agitation determination system 200 may be a system including the learning device. In this case, the determination device 220 determines an agitated state of the target patient using the agitation determination model generated by the learning device 10. The agitation determination system 200 may have a retraining function. The agitation determination system 200 generates the agitation determination model by the learning device 10 further using the biometric information of the target patient acquired by the target patient information acquisition unit 221 and the medical record information of the target patient acquired from a storage device, which is not illustrated, or the like. The agitation determination system 200 may perform retraining when the determination result of an agitated state of the target patient output by the determination device 220 does not reach preset predetermined accuracy. The determination device 220 in the agitation determination system 200 may include the configuration included in the learning device 10.

### Configuration of Hardware for Achieving Each Component of Example Embodiment

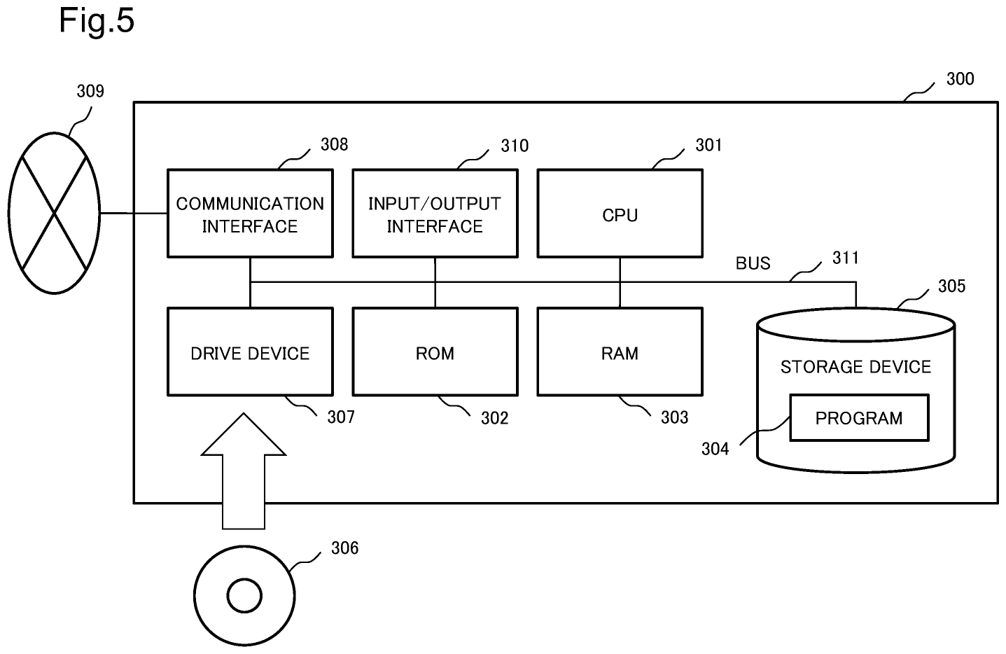

In each example embodiment of the present invention, each component of each device and system indicates a block in units of functions. Some or all of the components of each device and system are achieved by, for example, any combination of an information processing device 300 and a program as illustrated in FIG. 5. The information processing device 300 includes the configuration described below as an example.
Central processing unit (CPU) 301
Read only memory (ROM) 302
Random access memory (RAM) 303
Program 304 loaded into the RAM 303
Storage device 305 storing the program 304
Drive device 307 that reads and writes with respect to a recording medium 306
Communication interface 308 connected to a communication network 309
Input/output interface 310 for inputting/outputting data
Bus 311 connecting the components

Each component of each device according to each example embodiment is achieved by the CPU 301 acquiring and executing the program 304 that achieves their functions. The program 304 for achieving the function of each component of each device is stored in the storage device 305 or the RAM 303 in advance, for example, and is read by the CPU 301 as necessary. The program 304 may be supplied to the CPU 301 via the communication network 309, or may be stored in advance in the recording medium 306, and the drive device 307 may read the program and supply the program to the CPU 301.

There are various modifications for the method of achieving each device. For example, each device may be achieved by any different combination of the information processing device 300 and the program for each component. A plurality of components included in each device may be achieved by any combination of one information processing device 300 and the program.

Some or all of the components of the devices are achieved by a general-purpose or dedicated circuit including a processor or the like, or a combination thereof. These may be configured by a single chip or may be configured by a plurality of chips connected via a bus. Some or all of the components of the devices may be achieved by a combination of the above-described circuit or the like and the program.

In a case where some or all of the components of the devices are achieved by a plurality of information processing devices, circuits, and the like, the plurality of information processing devices, circuits, and the like may be arranged in a centralized manner or may be arranged in a distributed manner. For example, the information processing device, the circuit, and the like may be achieved as a form in which each is connected via a communication network, such as a client and server system or a cloud computing system.

In the above description, the example of generating the model for determining an agitated state of the patient has been described, but the invention of the present application is not limited to a model for determining an agitated state, but can be applied to any scene in which a model for determining the state of the subject such as a patient is generated.

While the invention of the present application has been described with reference to the example embodiments, the invention of the present application is not limited to the example embodiments described above. Various changes that can be understood by those of ordinary skill in the art can be made to the configuration and details of the invention of the invention of the present application within the scope of the present invention. The configurations of the above-described example embodiments may be combined or some constituent parts may be interchanged.

Some or all of the above example embodiments may also be described as the following supplementary notes, but are not limited to the following.

(Supplementary Note 1)

A learning device including
an acquisition means that acquires biometric information of a patient and medical record information of the patient,
a selection means that selects, based on the medical record information, the biometric information from which it is possible to determine whether the patient is in an agitated state, and
a model generation means that generates an agitation determination model for determining whether a target patient is in an agitated state based on the biometric information of the target patient by using the selected biometric information.

(Supplementary Note 2)

The learning device according to supplementary note 1, in which the selection means selects the biometric information of a patient in which information regarding a condition of the patient included in the medical record information satisfies a first condition regarding the condition of the patient, as the biometric information from which it is possible to determine whether of being in the agitated state.

(Supplementary Note 3)

The learning device according to supplementary note 2, in which the information regarding the condition of the patient includes information regarding a state of cognitive function or physical function of the patient.

(Supplementary Note 4)

The learning device according to supplementary note 2 or 3, in which the information regarding the condition of the patient includes at least one of a disease name, a disease state, a Glasgow Coma Scale (GCS) score, a Japan Coma Scale (JCS) score, and a Manual Muscle Test (MMT) score.

(Supplementary Note 5)

The learning device according to any one of supplementary notes 2 to 4, in which the first condition is a condition regarding a degree of the condition of the patient.

(Supplementary Note 6)

The learning device according to any one of supplementary notes 2 to 5, in which the biometric information from which it is possible to determine whether of being in the agitated state is the biometric information related to a patient having a low severity of the condition.

(Supplementary Note 7)

The learning device according to any one of supplementary notes 2 to 6, in which the biometric information from which it is possible to determine whether of being in the agitated state is the biometric information related to a patient capable of conscious movement or utterance.

(Supplementary Note 8)

The learning device according to any one of supplementary notes 1 to 7, in which the selection means selects biometric information of the patient in which information regarding a medical treatment action performed on the patient included in the medical record information satisfies a second condition regarding the medical treatment action as the biometric information from which it is possible to determine whether of being in the agitated state.

(Supplementary Note 9)

The learning device according to supplementary note 8, in which the medical treatment action includes at least any one of medication, treatment, restraint, and meal.

(Supplementary Note 10)

The learning device according to supplementary note 8 or 9, in which the second condition is a condition regarding a degree of variation in the biometric information caused by the medical treatment action.

(Supplementary Note 11)

The learning device according to any one of supplementary notes 8 to 10, in which the biometric information from which it is possible to determine whether of being in the agitated state is the biometric information in a time other than a time when the medical treatment action is performed on the patient.

(Supplementary Note 12)

The learning device according to any one of supplementary notes 8 to 10, in which the selection means selects the biometric information from which it is possible to determine whether of being in the agitated state by applying the second condition to the biometric information selected by applying the first condition to the medical record information.

(Supplementary Note 13)

A determination device including a determination means that determines whether the target patient is in the agitated state by using the biometric information of the target patient and the agitation determination model, in which the agitation determination model is a model generated by the learning device according to any one of supplementary notes 1 to 12.

(Supplementary Note 14)

A method for generating a trained model, including, by a computer
acquiring biometric information of a patient and medical record information of the patient,
selecting the biometric information from which it is possible to determine whether the patient is in an agitated state by using the medical record information, and
generating an agitation determination model for determining whether a target patient is in the agitated state based on the biometric information of the target patient by using the selected biometric information.

(Supplementary Note 15)

A recording medium storing a program for causing a computer to execute processing of
acquiring biometric information of a patient and medical record information of the patient,
selecting the biometric information from which it is possible to determine whether the patient is in an agitated state by using the medical record information, and
generating an agitation determination model for determining whether a target patient is in the agitated state based on the biometric information of the target patient by using the selected biometric information.

### REFERENCE SIGNS LIST

10 learning device
11 acquisition unit
12 selection unit
13 model generation unit
200 agitation determination system
220 determination device
221 target patient information acquisition unit
222 determination unit
223 output unit
230 biometric information acquisition device
240 determination result output device
300 information processing device
301 CPU
302 ROM
303 RAM
304 program
305 storage device
306 recording medium
307 drive device
308 communication interface
309 communication network
310 input/output interface
311 bus

What is claimed is:

1. A learning device comprising:
a memory storing instructions; and
at least one processor configured to execute the instructions to:
acquire biometric information of a patient and medical record information of the patient;
based on the medical record information, the biometric information from which it is possible to determine whether the patient is in an agitated state; and
generate an agitation determination model for determining whether a target patient is in the agitated state based on the biometric information of the target patient by using the selected biometric information.

2. The learning device according to claim 1, wherein
the at least one processor is further configured to execute the instructions to:
select the biometric information of a patient in which information regarding a condition of the patient included in the medical record information satisfies a first condition regarding the condition of the patient, as the biometric information from which it is possible to determine whether of being in the agitated state.

3. The learning device according to claim 2, wherein
the information regarding the condition of the patient includes information regarding a state of cognitive function or physical function of the patient.

4. The learning device according to claim 2, wherein
the information regarding the condition of the patient includes at least one of a disease name, a disease state, a Glasgow Coma Scale (GCS) score, a Japan Coma Scale (JCS) score, and a Manual Muscle Test (MMT) score.

5. The learning device according to claim 2, wherein
the first condition is a condition regarding a degree of the condition of the patient.

6. The learning device according to claim 2, wherein
the biometric information from which it is possible to determine whether of being in the agitated state is the biometric information related to a patient having a low severity of the condition.

7. The learning device according to claim 2, wherein
the biometric information from which it is possible to determine whether of being in the agitated state is the biometric information related to a patient capable of conscious movement or utterance.

8. The learning device according to claim 2, wherein
the at least one processor is further configured to execute the instructions to:
select the biometric information of a patient in which information regarding a medical treatment action performed on the patient included in the medical record information satisfies a second condition regarding the medical treatment action as the biometric information from which it is possible to determine whether of being in the agitated state.

9. The learning device according to claim 8, wherein
the medical treatment action includes at least any one of medication, treatment, restraint, and meal.

10. The learning device according to claim 8, wherein
the second condition is a condition regarding a degree of variation in the biometric information caused by the medical treatment action.

11. The learning device according to claim 8, wherein
the biometric information from which it is possible to determine whether of being in the agitated state is the biometric information in a time other than a time when the medical treatment action is performed on the patient.

12. The learning device according to claim 8, wherein
the at least one processor is further configured to execute the instructions to:
select the biometric information from which it is possible to determine whether of being in the agitated state by applying the second condition to the biometric information selected by applying the first condition to the medical record information.

13. A determination device comprising:
the at least one processor is further configured to execute the instructions to:
determine whether the target patient is in the agitated state by using the biometric information of the target patient and the agitation determination model, wherein
the agitation determination model is a trained model generated by the learning device according to claim 1.

14. A method for generating a trained model by a computer, comprising:
acquiring biometric information of a patient and medical record information of the patient;
selecting the biometric information from which it is possible to determine whether the patient is in an agitated state by using the medical record information; and
generating an agitation determination model for determining whether a target patient is in the agitated state based on the biometric information of the target patient by using the selected biometric information.

15. A recording medium non-transitorily storing a program for causing a computer to execute processing of:
acquiring biometric information of a patient and medical record information of the patient;
selecting the biometric information from which it is possible to determine whether the patient is in an agitated state by using the medical record information; and
generating an agitation determination model for determining whether a target patient is in the agitated state based on the biometric information of the target patient by using the selected biometric information.
